(19) United States
(12) Patent Application Publication
(10) Pub. No.: US 2011/0257571 A1
(43) Pub. Date: Oct. 20, 2011

(54) Post mountable BAHA bandage device

(76) Inventors: Michael H. Fritsch, Lincoln, NE (US); John H. Fritsch, Lincoln, NE (US); Josephine Fritsch, Lincoln, NE (US)

(21) Appl. No.: 13/066,775

(22) Filed: Apr. 25, 2011

Related U.S. Application Data
(63) Continuation-in-part of application No. 12/953,142, filed on Nov. 23, 2010.
(60) Provisional application No. 61/281,855, filed on Nov. 23, 2009.

(51) Int. Cl. A61L 15/44; A61L 15/22; A61F 13/00

(52) U.S. Cl. 602/46; 602/53; 602/48

(57) ABSTRACT

A bandage system is provided that is capable of being held by an upstanding snap member attached to a wound site. The bandage system has a snap engaging member for coupling to the snap member. A cushion member is provided that has a wound engaging surface for overlaying the wound site. A pressure exerting base member is coupled to the base member for urging the cushion member into engagement with the wound site.

### I. REFERENCE TO RELATED PATENT APPLICATIONS

This U.S. non-provisional patent application is a continuation in part of Michael Fritsch et al U.S. patent application Ser. No. 12/953,142, which was filed on 23 Nov. 2010, and claims the benefit of and/or priority to Fritsch et al., U.S. provisional patent application, Ser. No. 61/281,855 filed Nov. 23, 2009 entitled “BAHA (Bone-Anchored-Hearing-Aid) Bandage Device”, the entire contents of both of which applications are specifically incorporated herein by reference.

### II. TECHNICAL FIELD OF THE INVENTION

The present invention relates to bandage systems used in connection with surgically implanted hearing aid devices and more particularly to bandage systems used after the surgical implantation and skin grafting performed during the installation of a Bone-Anchored-Hearing-Aid (BAHA)™ device and other surgical procedures resulting in a post mount system.

### III. BACKGROUND OF INVENTION

A Bone-Anchored-Hearing-Device (BAHA™) is a device that allows the transmission of sound vibrations from a hearing aid to the skull and the inner ear. The hearing aid component is “snapped” or affixed onto the osteo-integrated post and crown (“abutment” and “fixture”) component. The vibrations travel through the post-crown component and vibrate the skull bone. The vibrations in the skull bone are conducted through the bone to the inner ear, where the vibrations trigger the hearing cascade.

The BAHA™ device is surgically implanted. As part of the surgical procedure, a skin graft is elevated. The soft tissue between the skin graft and the periosteal layer is removed . It is necessary to remove the soft tissue in order that the post-crown component rises above all tissues and allows the hearing aid component to be “snapped-on” to the post, and ‘snapped off’ the post when removal is desired. Also, it removes hair follicles from the site to allow for better hygiene and less entanglement. After the BAHA™ has been implanted, the skin graft is placed on to the periosteal layer and heals in that position.

The skin graft takes two to three weeks to attach and heal to the periosteum. During this time, a dressing must be placed over the wound to compress the skin graft onto the periosteum. Presently, circum-cephalic (around the head) type wraps, or a combination of adhesive bandages are used to compress the skin graft onto the periosteum. These wraps and bandages are very prone to falling off, to dislocating, and are difficult for the patient to maintain. The Cochlear corporation manufacturer of the BAHA supplies a “healing cap” that is about the size of a penny and is inadequate to sustain bandage dressings.

One object of the present invention is to provide a better dressing approach to overcome the difficulties with the known prior art bandages and wraps.

### IV. SUMMARY OF THE INVENTION

In accordance with the present invention, a bandage system is provided that is capable of being held by an upstanding post that is attached to the wound site. The bandage system includes a flexible retainer portion and a cushion member. The cushion member is placeable between the flexible retainer and the wound site for overlaying the wound site. The flexible retainer portion includes an aperture sized and positioned to receive the upstanding post, to permit the post to pass through the flexible member and the cushion member.

Preferably, at least one of the cushion member and the retainer portion include inwardly radiating, flexible petals surrounding the aperture, for engaging the upstanding post, for retaining the bandage on the post.

Additionally, the flexible petals should be sized so that they gripingly engage the post, so that the position of the petals and hence the flexible retainer member can be varied along the length of the post, to thereby enable the practitioner to vary the amount of compression exerted on the cushion member, to thereby vary the amount of compression exerted by the cushion member on the wound site.

In a most preferred embodiment, the cushion device includes a first end portion, a second end portion and a central portion that is disposed between the first and second end portions. The cushion member has a thickness that varies between the central portion first end portion and second portion, such that the cushion member is relatively thinner in its central portion, and relatively thicker as one moves outwardly. This increased thickness toward the edges of the cushion helps to enable the cushion member to better match the curvature of the skull.

Also in accordance with the present invention, a method is provided for bandaging a wound site at which an upstanding post has been attached to a body part at the wound site. The method first includes providing a bandage that includes a flexible retainer portion and a cushion portion, and an aperture extending through the flexible retaining portion and cushion portion. The aperture of the flexible retainer portion is defined by a plurality of radially inwardly extending gripping petals sized for grippingly engaging the upstanding post.

The upstanding post is then inserted through the aperture to place the cushion portion adjacent to the wound site. The upstanding post is then inserted through the petal defining apertures of the flexible retaining portion for engaging the flexible portion to the post, and to overlay the cushion portion for retaining the cushion portion in place over the wound site. The position of the petals of the flexible retaining portion is then adjusted on the upstanding post to thereby vary the distance between the flexible retainer portion and the wound site, to thereby adjust the degree of compression exerted by the flexible retainer portion on the cushion portion.

One feature of the present invention is that a bandage system is provided that includes a cushion member and a flexible retainer member that each include an aperture, that is sized and positioned for receiving an upstanding post attached to a wound site.

This feature has the advantage of providing a bandage that is attachable to a post, and positionable adjacent to a wound site, without the use of head wraps and wrap around bandages. Another advantage of the present invention is that by coupling the bandage system to the post, one does not need to use adhesives in a hair-filled area (the scalp). The use of such an adhesive against such hair can both cause pain when the bandage is removed, and also can increase the risk of possible infection and germs introduced by the hair.

Another feature of the present invention is that the aperture of the retaining portion is defined by a plurality of radially inwardly extending gripping petals that are sized for grippingly engaging the upstanding post.

This feature has the advantage of securely coupling the retaining portion, and hence, the bandage system to the post. Additionally, this feature has the advantage of enabling the user to vary the distance between the flexible retaining portion and the wound site. By varying the relative distance between the flexible retaining portion and the wound site, the user can vary the degree of compression exerted by the cushion member on the wound site.

An additional feature is that the gripping friction of engagement of the flexible petals permits the bandage, including the flexible retaining member and the cushion member, to be rotated about an axis defined by the axis of the upstanding post. Rotation about this axis allows the bandage to be rotated into and out of position. This ability to rotate into and out of position can be especially useful when a practitioner seeks to observe the wound site under the bandage without removing the bandage from its engagement with the upstanding post-type snap.

Another feature of the present invention is that although it has particular utility when used in connection with hearing aid-type devices, the device is also flexible enough in its potential uses to have utility in connection with other medical prosthetic and/or orthodontic applications wherein the need for a better bandage in the area around a surgically implanted post is desirable.

Also in accordance with a second embodiment of the present invention, a bandage system is provided that is capable of being held by an upstanding snap member attached to a wound site. The bandage system comprises a snap engaging member for coupling to the snap member. A cushion member is provided that has a wound engaging surface for overlaying the wound site. A pressure exerting base member is coupled to the base member for urging the cushion member into engagement with the wound site.

Preferably, the bandage system base member is coupled to the snap engaging member, and overlaps the cushion member. Further, the base member is preferably more rigid than the cushion member, so as to be able to exert a downward compressive force on the cushion member. In a most preferred embodiment, the base member can be “prestressed”, to be relatively more convex when the snap engaging member is not engaged to the snap member; and relatively less convex (and hence more planar) when the snap engaging member is coupled to the snap member.

One feature of the present invention is that the base member is provided that is capable of exerting a compressive force on the cushion member. This compressive force helps the underside, wound engaging surface of the cushion member to maintain its engagement with the wound site, to thereby facilitate healing through this engagement. Additionally, through the engagement of the underside surface of the cushion member with the wound site, the cushion member helps to better prevent foreign debris, such as germs, dirt and the like from coming in contact with the wound site.

Another feature of the present invention is that it employs a snap engaging member. A snap engaging member enables the bandage system to be engaged to a snap type post that might be coupled to a wound site. This snap engagement provides a positive, but yet selectively removable attachment mechanism between the bandage and the post. This removable attachability facilitates both the engagement of the cushion member of the bandage with the wound site, and also makes it easy to replace the bandage at regular intervals, as is consistent with appropriate wound care practices.

Another feature is that the cushion member can be made of foam or other material that can be impregnated with medications, balms, ointments and other substances to help the wound heal faster.

These and other features and advantages of the present invention will become apparent to those skilled in the art upon a review of the drawings and detailed description presented below, that represent the best mode of practicing the invention perceived presently by the applicants.

### V. BRIEF DESCRIPTION OF DRAWINGS

FIG. 1 is a first side sectional view of a bandage system mounted and in position covering a wound site;

FIG. 1A is an exploded, side sectional view of the bandage system of the present invention;

FIG. 2 is a top view of the flexible bandage retainer device;

FIG. 3 is a side view of the flexible bandage retention device and the cushion

FIG. 4 is a bottom view of the cushion device and the flexible bandage retaining device;

FIG. 5 is a top view of the device of the present invention, shown schematically as being applied to the patient;

FIG. 6 is a top view of the bandage system of the present invention, wherein the flexible retainer member is rotated 90° from its normal, in-place position;

FIG. 7 is a top view of the bandage system of the present invention covering a wound site;

FIG. 8 is a top view of the bandage system of the present invention covering a wound site, wherein the flexible retainer member is rotated 90° from its normal, in-place position;

FIG. 9 is a top view of the bandage system of the present invention covering a wound site, showing a ruler adjacent to the bandage system, to give a sense of scale to the device;

FIG. 10 is a perspective view of a first alternate embodiment bandage system of the present invention;

FIG. 11 is a sectional view taken along lines 11-11 of FIG. 10;

FIG. 12 is a side view of a pre-stressed bandage system of the present invention, showing the bandage in its “unstressed configuration”;

FIG. 13 is a side view, similar to FIG. 12, except showing the bandage 100 in engagement with a wound site, such that the bandage is in its “stressed” configuration;

FIG. 14 is a bottom view of the bandage system 100 of the present invention; and

FIG. 15 is a sectional view, similar to FIG. 11 of a second alternate embodiment of the present invention.

### VI. DETAILED DESCRIPTION

The device 10 of the present invention is shown in the figures as comprising a bandage assembly 10 which is designed to facilitate healing of the BAHA™ (Bone-Anchored-Hearing-Aid) surgical site. The device consists of two primary components, the “flexible retainer 24 and the cushion 28.

The device 10 is designed to be affixed to, and to encompass and cover the area around an upstanding post 12 such as is used with a BAHA™ device. The device 10 is used to compress a (wound site) skin graft into the periosteum PE around the BAHA™ device and surgical bed. This is done by the use of a central aperture 78 with inwardly radiating flexible petals 82 to clamp onto the columnar section 42 of the upstanding post 12. The compression force upon the (wound site) skin graft SG can be adjusted by raising or lowering the bandage device away from and towards the (wound site) skin graft SG through a sliding action of the radially inwardly facing surfaces 84 of the petals 78 along surface 44. This is useful for different lengths of upstanding posts 12.

Since the device 10 is held onto the column section 42 by radiating petals 82, it can be rotated about the axis “A” of the columnar section 42 so that the device 10 can reveal the underlying cushion or skin graft SG.

The bandage device 10, and particularly the flexible retainer 24 are made of materials that allow it to be trimmed by cutting with scissors to exactly fit the patients needs, such as the size and shape of the wound site SG. Alternatively, the device 10 can be manufactured in different sizes and shapes to fit the patients' needs.

The bandage device flexible retainer 24 is also curved in its longitudinal axis LA to provide for equal force of compression on the skin graft SG to compensate for the skull SK curvature. Alternately, the cushion 28 is made in a gradually increasing thickness so that the cushion becomes “wedge-shaped” from the central portion 54 of the cushion 28, adjacent to the aperture 26 to the periphery of the device adjacent first and second end portions 50, 52 to compensate for skull curvature away from a “flat shape” retainer 24.

The cushion 28 is designed to prevent water from entering under it (adjacent to underside surface 58) by being made of hydrophobic material. Also, in this way, any bleeding is vented away from under the Bandage Device.

The cushion 28 is designed to be impregnated or surface coated with substances and medications which will facilitate healing. The substances can be of ointments, gels, liquids, or powders. The size of bandage device can be varied, although only one size is shown in the figures.

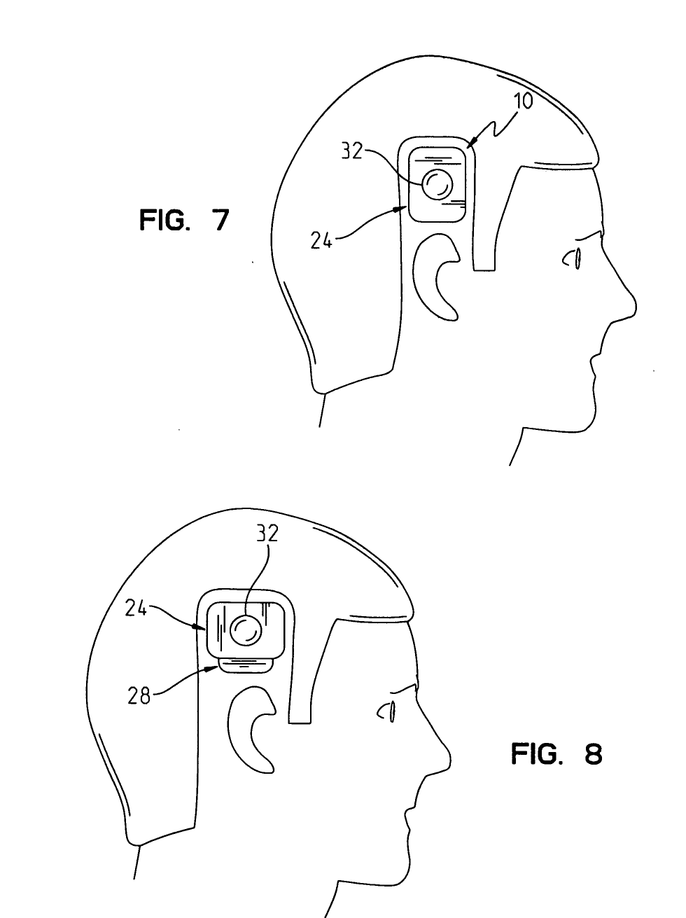

The bandage system 10 of the present invention is shown in the figures as being designed to be coupled to an upstanding post 12, of the type that is normally fixedly coupled to a skull during certain surgical procedures, such as a skin graft SG surgical procedure. The post 12 is attached to the skull SK to provide a vehicle for permitting a surgeon to couple a device, such as a hearing aid, to a body part BP, such as the skull SK of a user, in a manner wherein the device such as the hearing aid (not shown) can be selectively attached and removed from the body part BP 14 according to the needs and desires of the user. As shown in the drawings, the upstanding post 12 is coupled to the skull SK of the user. The skull SK includes a periosteum, to which a skin graft SG has been applied. As shown in FIGS. 6-8, it will noted that at the wound site, (shown as the skin graft SG area), the hair that is normally found on the scalp of the skull SK is shaved away and removed.

The bandage system itself comprises three primary components, including a sheet-like flexible retainer 24, a relatively thickened compressive cushion 28, and a healing cap 32, that can be coupled to the outer or proximal snap head portion 38 of the upstanding post 12. The upstanding post 12 includes a threaded bone-engaging distal end portion 36, having radially outwardly facing threads for engaging the boney skull SK of the user. The threads 36 are designed to threadedly engage the skull SK, to hold the post 12 in position in the skull SK so that it is disposed generally perpendicular to the plane of the surface of the skull SK.

The upstanding post 12 includes a snap-like head portion 38. The snap-like head portion 38 includes snap-like features so that the end cap 32, and the hearing aid device (not shown) can be snapingly engaged to the snap-like head portion 38 of the post 12 for coupling the snap-like head portion 38 and the post 12 to the hearing aid device (not shown).

A central shaft portion 42 extends between the snap-like head portion 38 and the threaded distal portion 36. The central shaft portion 42 is preferably cylindrical in configuration and includes a radially outwardly facing surface 44.

The cushion member 28 includes a body 49 having a first end portion 50 and a second end portion 52. A central portion 54 is disposed between the first end portion 50 and the second end portion 52. The cushion member 46 is generally thickened, somewhat similar to a thickened pad and includes an upper surface 56 and a lower surface 58. A central aperture 60 extends through the central portion 54 of the cushion member 28 between the upper surface 56 and the lower surface 58.

It will be noted in the drawings that the cushion member 28 is generally thinner in the area adjacent to the central aperture 60. As one moves from the central aperture 60 outwardly toward the respective first and second end portions 50, 52, the thickness of the cushion 28 member increases. This thickness increases to help accommodate the curvature of the scalp so that the lower surface 58 is more prone to engage the scalp.

A person's scalp is rarely flat but is rather an irregular sphere-shaped body, having a curvature. As shown in FIGS. 6-8, the bandage device 10 is designed normally to go on the side of the head, which has a slight curvature. The increasing thickness of the cushion 28 as one goes radially outwardly on the cushion 28 from the central aperture 60 toward the first and second ends 50, 52, helps to maintain an equally compressive force against the wound site, so that the compressive force exerted adjacent to the central aperture 60 is generally similar to the compressive force exerted adjacent to the first and second ends 50, 52 of the cushion 28.

The flexible member 24 comprises preferably a formed plastic-like sheet, that, while flexible, has sufficient rigidity, so that when pressed tightly against the cushion member 28, the flexible member 24 will have sufficient rigidity so that it can impart a desired shape to the upper surface 56 of the cushion member 28 rather than the cushion member 28 imparting a curve or surface characteristic to the flexible member 24. The flexible member 24 includes a first end portion 66 that is disposed adjacent to the first end portion 50 of the cushion member 28, and a second end portion 68 that is disposed adjacent to the second end portion 52 of the cushion member 28.

The flexible retainer member 24 also includes a central portion 70. The central portion 70 is disposed between the first and second end portions 66, 68. The flexible retainer member 24 also includes an upper surface 74 and a lower surface 76. The lower surface 76 is placed adjacent to the upper surface 56 of the cushion 26, and is designed to overlay the upper surface 56 and to engage the upper surface 56 of the cushion 26. The central portion 70 of the flexible retainer 24 includes a central aperture 78.

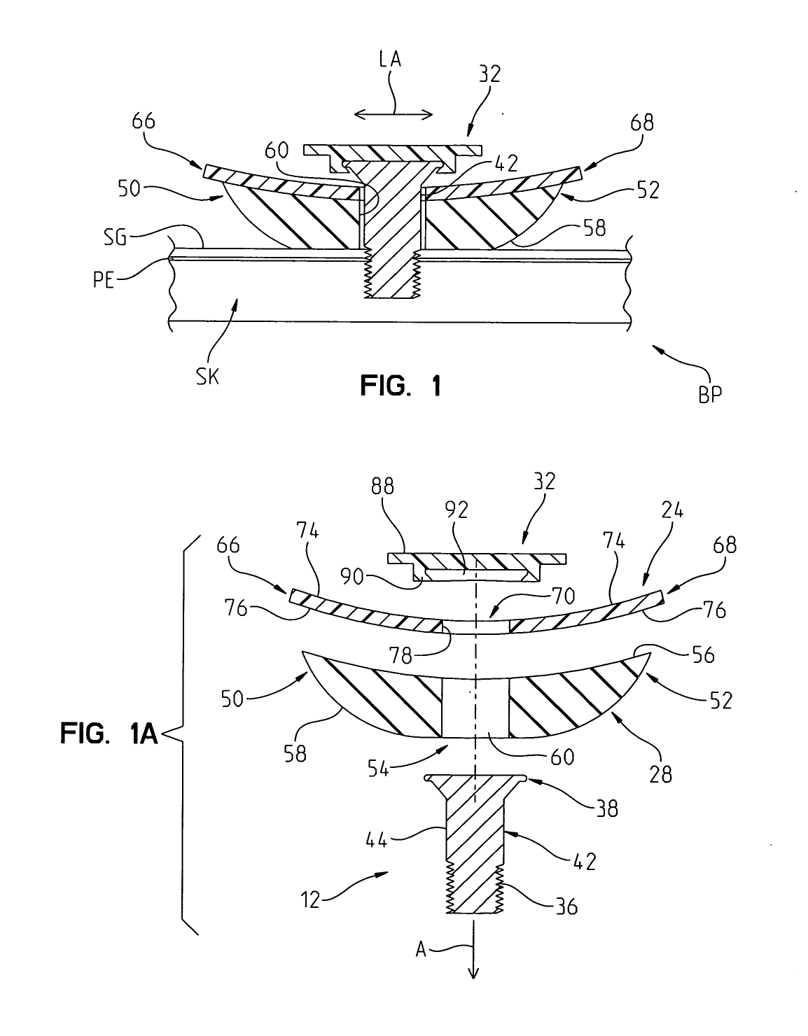

As best shown in FIG. 1A, the flexible retainer member 24 can have a curve along its longitudinal axis LA, such as the first and second ends 66, 68 are raised relative to the relatively depressed central portion 70 of the flexible retainer 24.

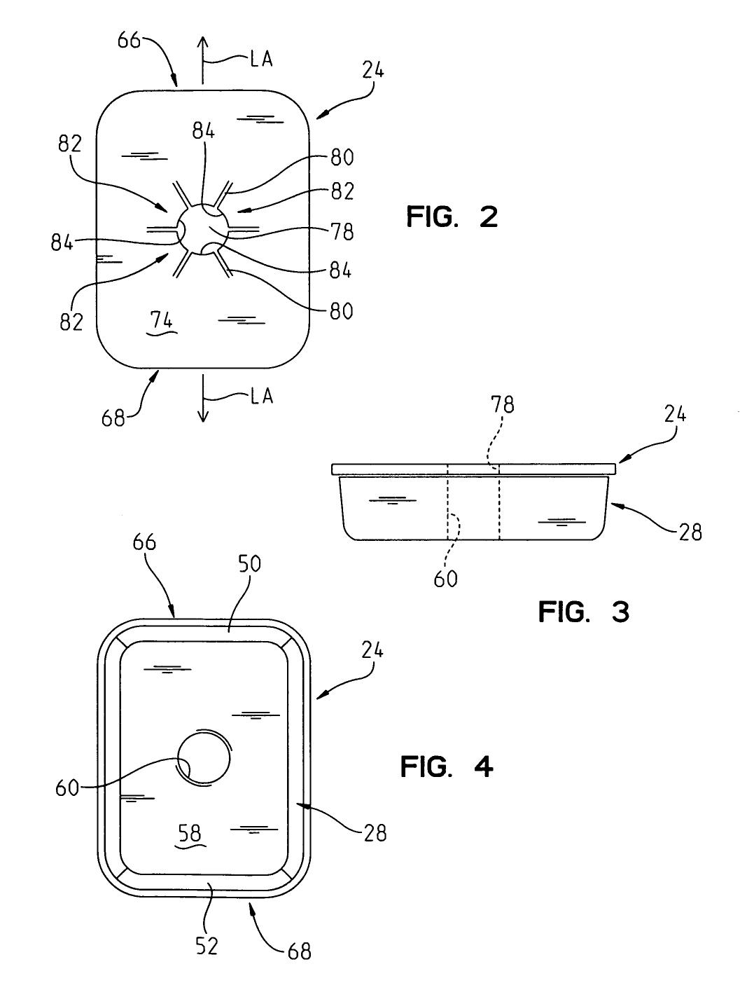

The central aperture 78 and its petals 82 are best shown in FIG. 2. In FIG. 2, it will be noted that the central aperture 78 of the flexible member 24 is defined by a series of radially inwardly extending flexible petals 82. The petals 82 are themselves defined by a series of radially extending slots 80 that are cut out between adjacent petals 82. Each of the petals 82 includes a radially inwardly facing surface 84, that is designed to frictionally engage the radially outwardly facing surface 44 of the central portion 42 of the upstanding post 12.

Through the frictional engagement of the radially inwardly facing surfaces 84 of the petals 82 with the post 12, the position of the flexible retainer member 24 can be adjusted longitudinally along the central shaft portion 42 of the upstanding post 12. By being able to vary the position along central shaft portion 42 at which the petals 82 and hence the flexible retainer portion 24 engages the central shaft 42, one can adjust the distance between the flexible retainer portion 24 and the wound site. This ability to vary the distance enables the user to vary the amount of compressive force exerted on the cushion 28 by the flexible retainer member 24. Additionally, the frictional engagement between the radially inwardly facing surfaces 84 of the petals 82 and the radially outwardly facing surface 44 of the central shaft portion 42 enables the flexible retainer 24 and hence the bandage device 10 to accommodate post 12 shafts 42 having different lengths.

The healing cap member 32 is designed to engage the snap member 38 at such times when the hearing aid (not shown) is not engaged to the snap member 38 of the upstanding post 12. Additionally, the healing cap 32 is coupled to the snap portion 38 of the upstanding post 12 after the surgery is completed, and before the hearing aid is being worn regularly, during such times as when the wound site is healing. The healing cap 32 comprises a top or upper grabbable portion 88 and a lower, snap receiving portion 90. The snap receiving portion 90 includes a cavity 92 for receiving the upper snap portion 38 of the upstanding post 12, and a downwardly extending portion that defines the central aperture 92. The healing cap 32 can be snappingly engaged to the post 12.

Returning now to all of the figures, the method is described by which the bandage 10 is used to bandage a wound site SG at which an upstanding post 12 has been attached to a body part BP. First, a bandage 10 having a flexible retainer portion 24, a cushion portion 26 and an aperture 78 that extends through the respective flexible retaining portion 24, and cushion 28 is provided. The aperture 78 of the flexible retaining portion 24 is defined by a plurality of radially inwardly extending gripping petals 82 that are sized for grippingly engaging the upstanding post 12.

The upstanding post 12 is inserted through the aperture 60 of the cushion member 28 to place the cushion member 28 adjacent to the wound site SG. The upstanding post 12 is then inserted through the petal defining aperture 78 of the flexible retaining portion 24 for engaging the flexible retaining portion 24 to the post 12, and to overlay the cushion portion 28 for retaining the cushion portion 28 over the wound site SG. The petals 82 of the flexible retaining portion 24 are adjusted on the upstanding post 12 to thereby adjust the distance between the flexible retaining portion 24 and the wound site SG to thereby adjust the degree of compression exerted by the flexible retainer portion 24 on the cushion portion 28. Preferably, a healing cap member 32 having its snap receptacle portion 90 is snappingly coupled to the upstanding post 12, for maintaining the flexible retaining portion 24 and cushion portion 28 on the upstanding post 12.

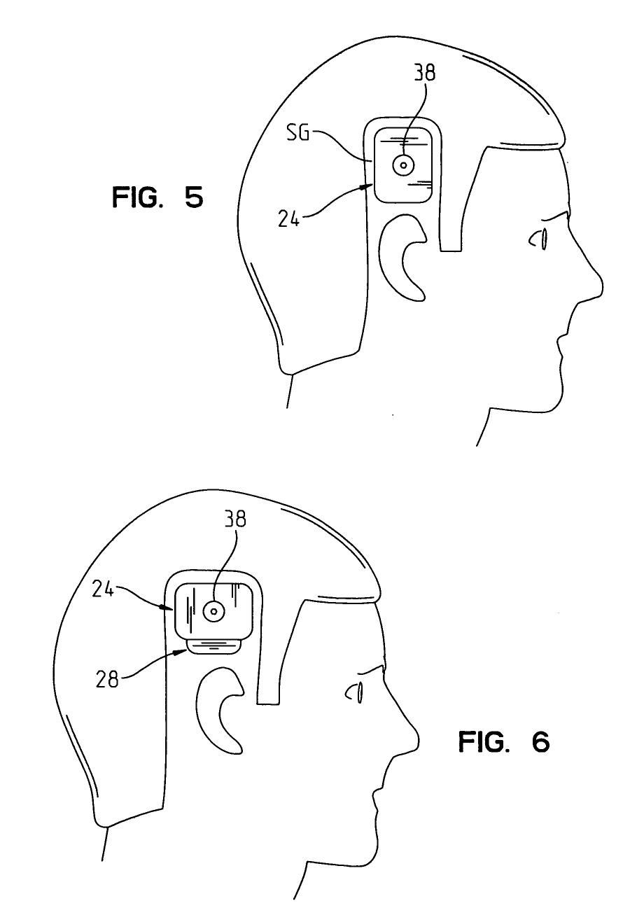

Turning now to FIGS. 5-9, the bandage device 10 is shown as being placed on the head of a patient. As shown in FIG. 5, the patient's head includes eyes, nose, mouth and an ear. As the bandage device 10 is preferably used to protect a skin graft-type wound site used in connection with a hearing aid, the wound site SG is positioned adjacent to the ear of the patient.

In order to perform the skin graft SG, hair is removed from the scalp adjacent to, the skin graft site SG, so that the skin graft site area is hairless. The skin graft is then performed. As part of this procedure, the post 12 is threadedly engaged into the bone of the skull, so that the post 12 is fixed with respect to the skull SK. The cushion member 28 is then placed over the post 12, as is the flexible retainer 24.

When in an appropriate position, the flexible retainer 24 overlays the cushion member 28, as shown in FIG. 5, so that the cushion member is not visible.

Turning now to FIG. 6, it will be noted that the flexible retainer 24 is rotated 90° about the axis of the post 12, and snap member 38. When so rotated, the viewer can see the exposed cushion member 28 and determine its condition. Typically, determining the condition helps the user or medical practitioner determine when and if the cushion member 28 needs to be replaced.

FIG. 7 is a view generally similar to FIG. 5. However, in FIG. 7, the device 10 is shown as being attached to a user, such that a healing cap 32 is placed over the snap member 38. As discussed above, this placement of the healing cap 32 over the snap member 38 helps to retain the bandage 10 on the post 12.

FIG. 8 is generally similar to FIG. 7, except that the healing cap 32 is placed on the snap member 38. The flexible retainer member 24 is rotated 90° to expose the underlying cushion member 28 similar to the manner in which the cushion member 28 is exposed in FIG. 6.

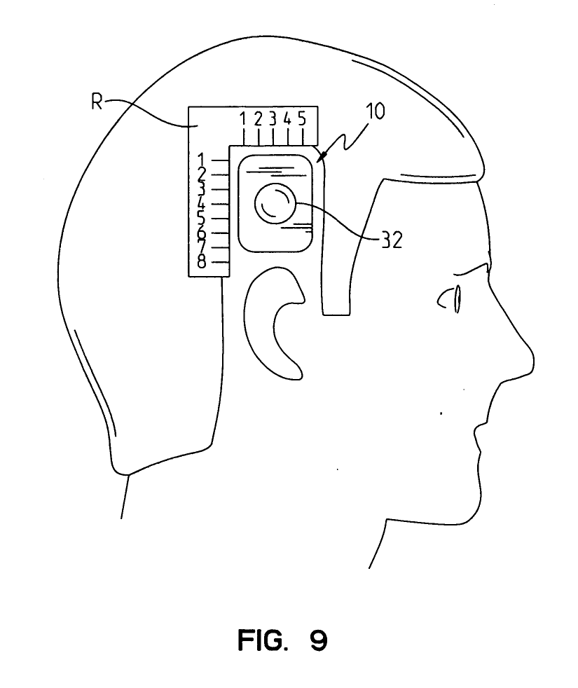

Turning now to FIG. 9, a view is shown that is similar to the view shown in FIG. 7, except that a ruler R is placed adjacent to the bandage to give the viewer some degree of scale. It will be noted that the bandage 10 shown in FIG. 9 has a width of approximately 4 cm, and a length of approximately 7 cm.

It will be understood that the exact area and dimensions of the bandage 10 will vary based both on the size of the user and also on the size of the wound graft or skin that the bandage is covering. This variance in size can be accomplished by either making the flexible member 24 and/or cushion member 28 out of a scissor-cuttable material so that the user can trim the flexible retainer 24 and/or cushion member 28 on site; or alternately, the bandage can be provided in a variety of pre-cut sizes.

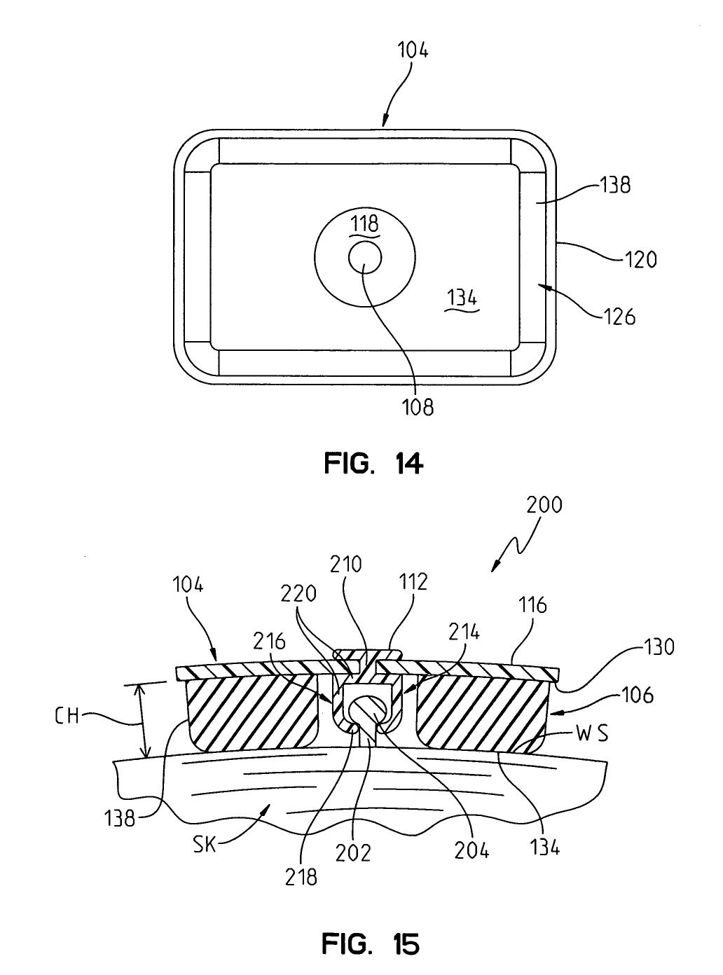

Turning now to FIGS. 11-14, a first alternate embodiment bandage system 100 is shown. Alternate bandage system 100 includes a snap engagement for snapping onto an upstanding post 111 that is attached to a wound site WS. The bandage system 100 includes a snap engaging member 108 for coupling to the upstanding post-type snap member 102 that is affixed to the wound site WS. A cushion member 106 has a wound engaging surface 134 for overlaying the wound site WS. A pressure exerting base member 104 that preferably comprises a stiffener type base member, is coupled to the cushion member 106 for urging the cushion member 106 into engagement with the wound site WS.

The cushion member 106 can be constructed similar to cushion member 28, such that cushion member 106 is made ina gradually increasing thickness as one moves from its central portion to its portion so that the cushion becomes wedge-shaped to compensate for skull SK curvature away from a generally flat or planar base portion 104. As will be discussed in more detail below, the cushion member 106 shown in the drawings has a generally constant thickness to accomodate a pre-stressed base member 104.

The snap engaging member 108 is coupled to the snap member 110 that comprises a radially inwardly extending portion that is formed at the distal end of the upstanding post 111.

The base member 104 is preferably sheet-like in configuration and is made from a generally rigid material, such as a rigid plastic. The base member 104 includes an upper outwardly facing surface 116, a lower surface 118, and a side edge portion 120.

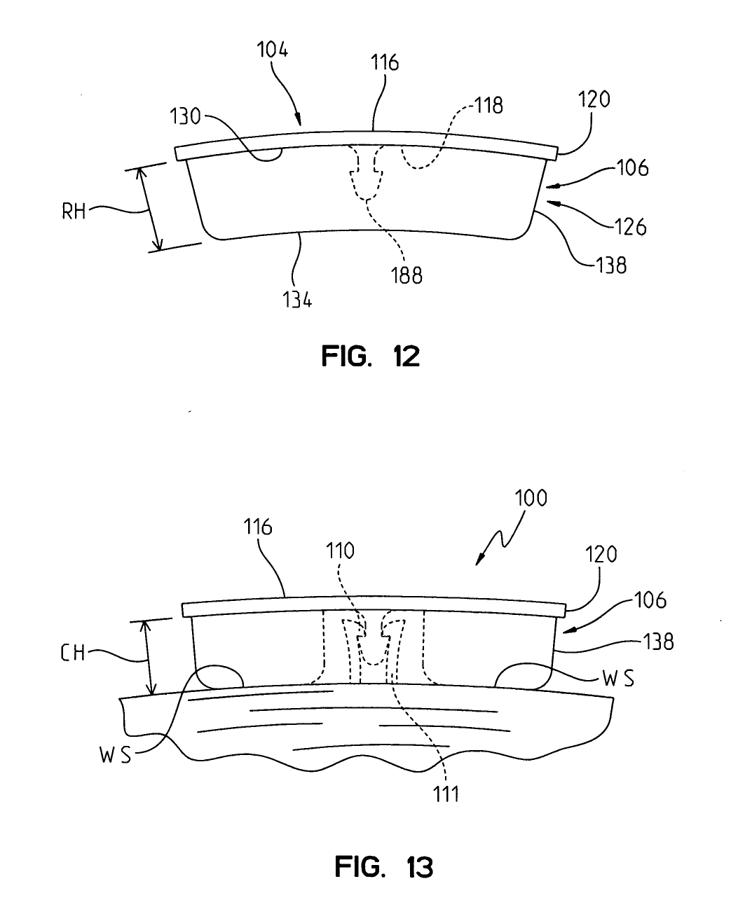

As shown in FIG. 13, the base member 104 can be formed as a “pre-stressed” member, so that when formed, it has something of a convex outer surface 116. The convex outer configuration shown in FIG. 13, can comprise the “relaxed” configuration of the base member 104. The base member 104 is generally more rigid and stronger and less flexible than the cushion member 106. The upper surface 130 of the cushion member is adhesively attached to the lower surface 118 of the stiffener member 116, generally along a substantial portion of the area of the respective upper surface 130 of the cushion member 106 and lower surface 118 of the base member 104. In view of this, the relatively rigid base member 104 imparts a somewhat convex configuration to the cushion member 106.

The cushion member 106 is preferably made from an absorbent gauze-like material typical of the type of wound treatment material that one would use with other bandages. Although such wound dressing materials for years have been made out of various cotton and cotton gauze materials, recent advances in technology have now resulted in a host of other materials being used for bandaging materials. Some of these materials are impregnated with various antibiotics, dressings, lotions, salves, cleansing agents and healing agents to aid in the healing process.

The cushion member 106 includes the upper surface 130, which as described above, is adhesively attached to the lower surface 116 of the base member; and the lower, wound engaging surface 134 that is disposed in an opposed relationship with the upper surface 130, and is designed and configured for engaging the wound site WS. A side surface 138 extends between the upper surface 130 and the lower surface 134.

The cushion member 106 is designed to be compressible in nature. Turning now to FIGS. 12 and 13, it will be noted that the cushion member 106 has a height RH, that relates to the “relaxed height” of the cushion member 106 when the cushion member 106 is in a relaxed state. This relaxed height is generally greater than the compressed height (FIG. 13) that exists when a cushion member is in a compressed position.

As best shown with reference to FIGS. 12 and 13, when the bandage 100 is not attached to a snap member 111 at a wound site WS, the cushion member 106 is uncompressed, and can expand to its full height, which comprises the relaxed height. However, because the relaxed height RH is generally greater than the distance between the underside surface 116 of the base 104 and the wound site WS, when the snap engaging member 108 is engaged in the snap member 110, the engagement of the snap engaging member 108 to the snap member 110 of post 111 causes the cushion member 106 to compress so that its height is reduced to the compressed height CH from the relaxed height RH.

Additionally, it will be noted that the formerly convex base member 120 becomes more planar, when the snap engagement member 108 is engaged with the snap member 110.

The pre-stressed generally convex nature of the base member 104 causes generally greater pressure to be placed on the cushion member 106 adjacent to the edge surfaces 138 of the cushion member 106. By so doing, this helps to keep the edges of the lower surface 134 of the cushion member 106 in engagement with the wound site WS, and helps to foster a good seal between the lower surface 134 of the cushion member 106 an the wound site WS, to prevent germs, dirt and other foreign matter from entering the area between the cushion member and the wound site, and thus possibly infecting the wound, or at least slowing the healing process.

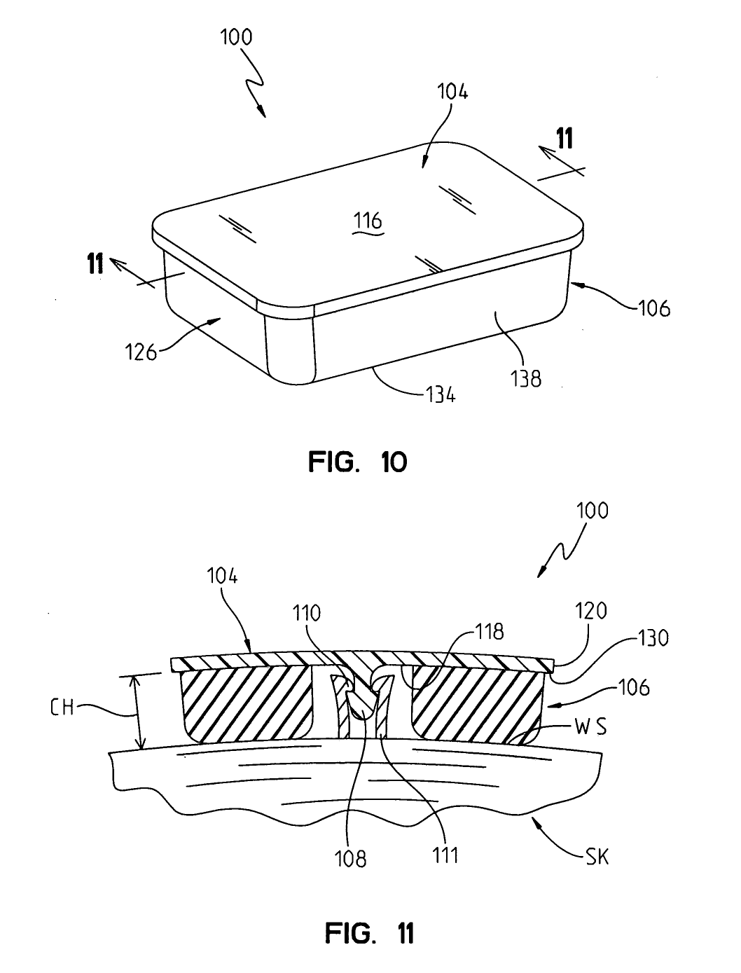

In the embodiment shown in FIGS. 10-14, it should be noted that the snap member 111 is generally a female snap member, such that the upstanding post 111 and snap member 110 comprise a socket having radially extending lips that help hold the generally male snap engaging member 108 within the post 111. Preferably, the snap engaging member 108 is sufficiently formable, so as to allow it to move through the opening defined by the snap member 110, to enter into the interior of snap member 110.

The male snap engaging member 108 is shown as unitarily formed to the base member 116. Such unitary and integrated snap member 106 can be formed through an injection molding process. Alternately, the snap engaging member 108 can be designed as a separate member that is then attached to the base member 104 in a secondary operation.

A second alternate embodiment 200 is shown in FIG. 15. The bandage system 200 embodiment shown in FIG. 15 has two primary differences between itself 200 and the embodiment 100 shown in FIGS. 10-14. The first difference is that the snap engaging member. 208 that is attached to the base member 104 is a separately formed female snap engaging member that is designed to interiorly receive the male snap member post 202 that is coupled to the wound site WS.

The male snap member post 202 includes a bulbous, mushroom-shaped head 204 that is interiorly received between the radially inwardly extending lips 218 that are disposed at the distal end of the cylindrical socket 214 of the female snap engaging member 208.

The female snap engaging member 208 of the embodiment shown in FIG. 15 also differs from the embodiment shown in FIG. 10. The female snap engaging member 208 comprises a separately manufactured item, that is attached to the base member 104 in a secondary operation. The female snap engaging member 208 includes a post 210 that extends in a direction through the base member 104 between the lower surface 130 and upper surface 116 of the base member 104.

A head member 212 includes an upper surface and a lower surface. The lower surface of the head member 212 engages the upper surface 116 of the base member 104. The head member 212 generally has a diameter that is greater than the diameter of the post 210 to help maintain the female snap engaging member 208 from becoming disconnected from the base member 104, because of the member sliding through the aperture of the base member 104 through which the post 210 extends.

The socket 214 also includes an axially extending cylindrical portion that terminates at its distal end in a circumferential radially extending lip 218, that is provided for engaging and holding the bulbous head 204 of the male post member 202. The socket further includes a radially extending base 220 having an upper surface that engages the lower surface 130 of the base member 104 and also has a diameter generally greater than the aperture through which the post 210 passes to help secure the female snap engaging member 208 on the base member, in conjunction with the head 212.

Other than the differences discussed above, the remaining components of the bandage system 200 can be constructed similarly to their counter parts shown in FIGS. 10-14.

Having described the invention with reference to certain details and preferred embodiments, it will be appreciated that variation and modifications exist within the scope and spirit of the present invention, as defined by the claims set forth below.

What is claimed:

1. A bandage system capable of being held by an upstanding snap member attached to a wound site comprising
(a) a snap engaging member for coupling to the snap member;
(b) a cushion member having a wound engaging surface for overlaying the wound site; and
(c) a pressure exerting base member coupled to the cushion member for urging the cushion member into engagement with the wound site.

2. The bandage system of claim 1 wherein the base member is coupled to the snap engaging member, and overlays the cushion member, and wherein the base member is relatively more rigid than the cushion member.

3. The bandage system of claim 2 wherein the base member is relatively more convex when the snap engaging member is not engaged to the snap member, and is relatively less convex when the snap engaging member is coupled to the snap member.

4. The bandage system of claim 2 wherein the base member comprises a base stiffener member that becomes stressed into a relatively more planar configuration when the snap engaging member is coupled to the snap member.

5. The bandage system of claim 4 wherein the cushion member is compressible and wherein the stress of the base member exerts a compressive force on the cushion member to maintain the wound engaging surface of the cushion member in engagement with the wound site.

6. The bandage system of claim 2 wherein the base member is configured to be positioned at a predetermined distance above the wound site when the snap engaging member is coupled to the snap member.

7. The bandage system of claim 6 wherein the cushion member has a relaxed height greater than the predetermined distance at which the base member is configured to be positioned above the wound site, wherein the base member exerts a compressive force against the cushion member to maintain the wound engaging surface of the cushion member in engagement with the wound site.

8. The bandage system of claim 1 wherein the base member is generally sheet-like in configuration and includes a first surface and a second surface, and the snap engaging member extends in a direction generally perpendicular and outwardly from the second surface.

9. The bandage system of claim 8 wherein the snap engaging member is unitarily formed with the base member.

10. The bandage system of claim 9 wherein the second surface of the base member engages an upper surface of the cushion member.

11. The bandage system of claim 10 wherein the upper surface of the cushion member is adhesively coupled to the second surface of the base member.

12. The bandage system of claim 1 wherein the cushion member has a central portion and an edge portion, and wherein the cushion is thicker in the edge portion then in the central portion.

13. The bandage system of claim 1 wherein the cushion member has a thickness that increases from a central portion of the cushion member to an edge portion of the cushion member.

14. The bandage system of claim 1 wherein the cushion member is impregnated with at least one of a medication, ointment and a balm.

15. The bandage system of claim 1 wherein the cushion member comprises a porous foam member impregnated with at least one of a medication and balm for promoting faster healing.
